(19) United States
(12) Patent Application Publication
(10) Pub. No.: US 2009/0131920 A1
(43) Pub. Date: May 21, 2009

(54) STENT CRIMPING APPARATUS

(75) Inventor: JAVIER PALOMAR MORENO, GALWAY CITY (IE)

Correspondence Address: FULWIDER PATTON, LLP (ABBOTT), 6060 CENTER DRIVE, 10TH FLOOR, LOS ANGELES, CA, 90045, US

(73) Assignee: ABBOTT LABORATORIES VASCULAR ENTERPRISES LIMITED, Dublin 2 (IE)

(21) Appl. No.: 11/941,683

(22) Filed: Nov. 16, 2007

(51) Int. Cl. A61B 17/00

(52) U.S. Cl. 606/1

(57) ABSTRACT

A device for crimping stents onto a catheter is described, comprising two opposing pressure walls. The walls are angled towards each other and provide surfaces that move in opposite directions so that a stent on a catheter placed between the surfaces is rotated while a force is applied to the stent. The stent is moved toward a narrower dimension between the pressure walls, to emerge from the walls fully crimped onto the catheter.

### BACKGROUND OF THE INVENTION

This invention relates to a stent or vascular prosthesis crimping device and method of use of the type that will enable a user to firmly crimp a stent onto the distal end of a catheter assembly.

In a typical percutaneous transluminal coronary angioplasty (PTCA) procedure, for compressing lesion plaque against the artery wall to dilate the arterial lumen, a guiding catheter is percutaneously introduced into the cardiovascular system of a patient through the brachial or femoral arteries and advanced through the vasculature until the distal end is in the ostium. A guidewire and a dilatation catheter having a balloon on the distal end are introduced through the guiding catheter with the guidewire sliding within the dilatation catheter. The guidewire is first advanced out of the guiding catheter into the patient's coronary vasculature, and the dilatation catheter is advanced over the previously advanced guidewire until the dilatation balloon is properly positioned across the lesion. Once in position across the lesion, a flexible, expandable, preformed balloon is inflated to a predetermined size at relatively high pressures to radially compress the atherosclerotic plaque of the lesion against the inside of the artery wall and thereby dilate the lumen of the artery. The balloon is then deflated to a small profile, so that the dilatation catheter can be withdrawn from the patient's vasculature and blood flow resumed through the dilated artery. While this procedure is typical, it is not the only method used in angioplasty. Further, other methods are well known in opening a stenosed artery such as atherectomy devices, plaque dissolving drugs, and the like.

In angioplasty procedures of the kind referenced above, there may be restenosis of the artery, which may require another angioplasty procedure, a surgical bypass operation, or some method of repairing or strengthening the area. To reduce the chance of restenosis and strengthen the area, a physician can implant an intravascular prosthesis for maintaining vascular patency, typically called a stent. A stent is a device used to hold tissue in place or to provide a support for a graft or tissue joined together while healing is taking place. A variety of devices are known in the art for use as stents, including coiled wires and wire mesh sleeves, in a variety of patterns, that are able to be crimped onto a balloon catheter, and expanded after being positioned intraluminally on the balloon catheter, and that retain their expanded form. Typically the stent is mounted and crimped onto the balloon portion of the catheter, and advanced to a location inside the artery at the lesion. The stent is then expanded to a larger diameter, by the balloon portion of the catheter, to implant the stent in the artery at the lesion.

However, if the stent is not tightly crimped onto the catheter balloon portion, when the catheter is advanced in the patient's vasculature the stent may slide off the catheter balloon portion in the coronary artery prior to expansion, and may block the flow of blood, requiring procedures to remove the stent.

In procedures where the stent is placed over the balloon portion of the catheter, the stent must be crimped onto the balloon portion to prevent the stent from sliding off the catheter when the catheter is advanced in the patient's vasculature. In the past, the crimping procedure was often done by hand, which may result in uneven force being applied, resulting in non-uniform crimps. In addition, it was difficult to judge when a uniform and reliable crimp had been applied or if the stent had damaged the balloon.

Since then, some tools have been developed for mechanically crimping a stent onto a catheter, but many of these suffer from shortcomings. For example, it may be difficult to terminate the crimping process at precisely the correct stent diameter. It may be difficult to achieve the correct amount of crimping without damaging the balloon. For example, many tools for crimping a stent onto a balloon will apply a purely radially inward force to the stent. To achieve the desired final diameter of the stent in these circumstances, the stent must be initially crimped to a radius substantially smaller than the desired final diameter. This arises because to achieve a permanent elastic deformation in the stent, the stent must be crimped beyond its elastic limit. However, even where plastic deformation is achieved, a substantial elastic rebound may take place after plastic deformation has been achieved. Thus, in the prior art, the stent must be initially deformed to a degree that may damage the balloon before elastic rebound takes place to leave the balloon with a desired final crimped diameter.

There accordingly remains a need for an improved tool and method for crimping a stent on a balloon. The present invention addresses these and other needs.

### SUMMARY OF THE INVENTION

This invention is directed to a vascular prosthesis crimping device which enables substantially uniform and tight crimping of a stent onto a catheter balloon portion, to better secure the stent onto the catheter for delivery of the stent through the patient's vasculature, while at the same time permitting uniform expansion of the stent in a patient's artery, vein, duct, or other vessel or lumen. The present invention attempts to solve several problems associated with crimping stents onto balloon catheters.

In a preferred embodiment, the device for crimping a stent mounted on a catheter comprises at least one pressure wall including two rotatable rods spaced apart and parallel. A flexible belt is looped around the rods, the belt being configured to provide a planar surface and to rotate about the rods when at least one of the rods is rotated under power.

In a further aspect of the invention, the device further includes a reinforcing block having a planar surface, positioned within a perimeter of the belt, for applying to the stent a force exerted perpendicular to a plane of the belt. Preferably, the reinforcing block is attached to a supporting element for holding the reinforcing block stationary relative to the crimping device, while the position of the reinforcing block relative to the device may be adjustable, and the rods may be held a fixed distance apart by at least one separation element. In another aspect of the invention, the at least one separation element is two separation elements, each separation element positioned on either side of the belt, and for which the length of the separation element is adjustable via a threaded turnbuckle for adjusting the length of the separation element and, thus, the amount of separation between the rods. The pressure walls are configured to apply an inward and also a rotating force to a stent placed between the walls, whereby a stent may be crimped onto a balloon portion of a catheter.

In yet a further aspect of the invention, the device has two pressure walls, that are not parallel to each other, but are angled toward each other and do not contact each other. In this aspect, the pressure walls are preferably mirror images of each other about a line of symmetry. In this aspect, the belts of the two pressure walls are configured to rotate simultaneously in the same direction, preferably clockwise and, alternatingly, anticlockwise. Each pressure wall is configured to apply a generally radially inward force on the stent, although it will be appreciated that, when the stent is rotated between the pressure walls a force vector is also applied that is in the direction of a tangent to the stent circumference. This tangential force is beneficial in that it allows elements of the stent to plastically deform without being forced radially inwardly, where the element may injure the integrity of the balloon. Thus, the resultant plastic deformation of the stent allows it to be crimped to a desired diameter without initially being deformed to a diameter less than the desired diameter.

The present invention also includes, in another facet, a method of crimping a stent onto a catheter. The method comprises mounting a stent in uncrimped condition onto a catheter. Thereafter, the stent is introduced between two opposing walls, each wall having a planar surface angled toward the other surface such that the distance between the surfaces at a first edge is less than the distance between the surfaces at an opposite second edge. The planar surfaces are caused to move in opposite directions to each other so as to cause the stent to roll between the walls, whereafter the stent is moved toward the first edge of the walls whereby the stent engages with the surfaces and is rotated. At the same time that the stent is rotated, the catheter is also rotated so that the stent and the catheter do not rotate in relation to each other while the stent is being moved toward the first edge. Following this method, the stent becomes radially compressed and crimped onto the catheter without initially being crimped to a diameter smaller than the desired final diameter.

In another aspect of this facet of the invention, causing the planar surfaces to move in opposite directions to each other includes rotating a belt on each of the walls in the same rotational direction. It will be appreciated that by rotating two belts in the same direction, surfaces of the belt facing each other will be moving in opposite directions, one surface will move upwards while the other will move downwards. Preferably, rotating a belt on each of the walls in the same rotational direction includes rotating the belt on each of the walls clockwise and, alternatingly, anticlockwise so that a reversal of the direction in which the stent is rotated takes place periodically.

In yet a further aspect of this facet of the invention, moving the stent in relation to the walls includes threading a guidewire through a central lumen in the catheter, and then moving the guidewire and the catheter in relation to the walls so that the stent follows the movement of the catheter. To facilitate this action, threading a guidewire through a central lumen of the catheter may be followed by placing the guidewire under tension, and holding the guidewire under tension when the guidewire and catheter are moved in relation to the walls. It will be appreciated that the guidewire under tension will have significantly more rigidity than when under no tension, and will facilitate the even movement of the catheter with mounted stent between the pressure walls.

These, and other features of the invention, will be disclosed more fully in the detailed description of the preferred embodiments that follow, and the drawings attached.

### BRIEF DESCRIPTION OF THE DRAWINGS

FIG. 1 is a perspective view of a stent crimping device having features of the present invention.

FIG. 2 is a top view of the device of FIG. 1.

FIG. 3 is a side sectional view of the device of FIG. 1.

FIG. 4 is an end sectional view of the device of FIG. 1.

### DETAILED DESCRIPTION OF THE PREFERRED EMBODIMENTS

With reference to the drawings, which are provided by way of exemplification and not limitation, a stent crimping device is described having features of the present invention. In a preferred embodiment, a stent crimping device, generally identified by the numeral 20, includes two mirror image pressure walls 22, 22′ positioned to oppose each other. Each pressure wall is configured to impart a radially inward force, and also a rotational force, on a stent located upon a catheter, as more fully set forth herein.

Each pressure wall includes an upper cylindrical bar 28, 28′ and a lower cylindrical bar 30, 30′. Each one of the four bars is configured to rotate upon its axis. In a preferred embodiment, one set of bars (the upper bars 28, 28′ or the lower bars 30, 30′) may be rotated under the power of a motor (not shown), although in an alternative embodiment both sets of bars may be rotated under power. Further included in each pressure wall, a flexible belt 26, 26′ is tightly looped around the upper and lower bars, such that powered rotation of at least one set of the upper or lower bars causes the belt to rotate under frictional force, and also causes a bar that may not be under power to rotate with the belt.

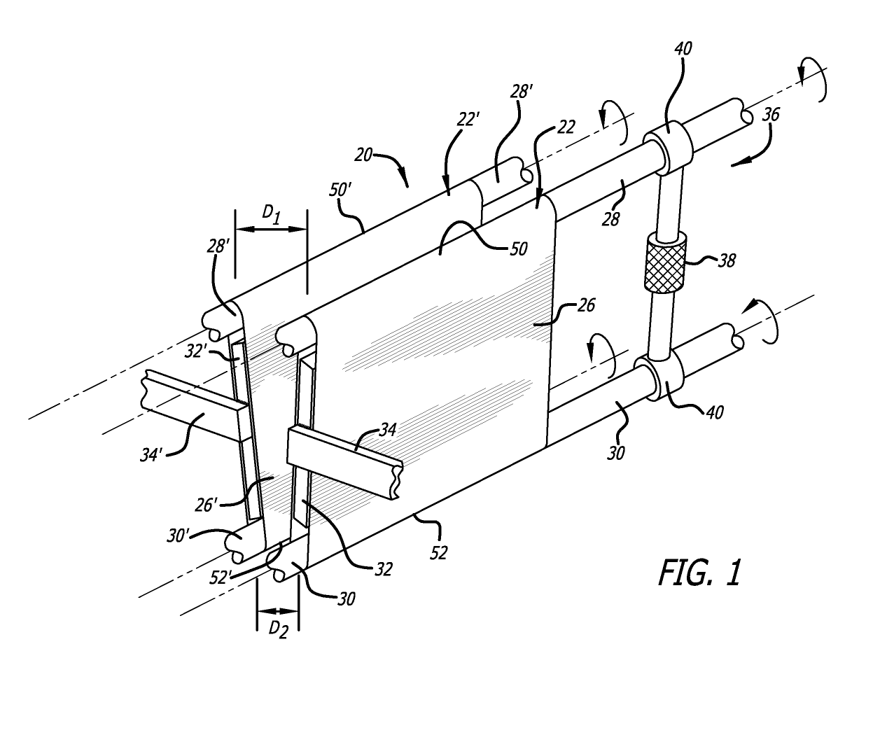

As seen in FIGS. 1 and 4, the pressure walls are not parallel, but incline slightly toward each other at one end so that the distance between the upper bars, D1, is larger than the distance between the lower bars, D2. Opposing bars that are driven are made to rotate in the same direction, so that the belts 26, 26′ are both driven clockwise or anticlockwise, or, alternatingly, clockwise followed by anticlockwise. This aspect will tend to advance an aim of the invention, as set forth more fully below.

Within the perimeter of each belt a reinforcing block 32, 32′ may be positioned, each block having parallel opposed outer surfaces and being dimensioned to allow the belt to slide snugly over the outer surfaces. Each block may be held motionless in relation to the overall device by a supporting element 34, 34′ which extends between each block and a stationary fixed object (not shown), and which permits the blocks to be spacially repositioned in relation to the rest of the device if necessary.

In order for the belts 26, 26′ to optimally rotate without slipping, the upper bars 28, 28′ may be held a fixed distance from the lower bars 30, 30′ by means of at least one separation element 36 configured to adjustably set the distance between upper and lower bars. As seen in FIG. 1, each separation element 36 includes upper and lower eyes 40 configured to snugly hold the bars apart from each other, but to permit rotation of the bars within an opening in the eye. The separation element 36 includes a length adjustment element 38, which, in a preferred embodiment as shown in FIG. 1, includes a threaded turnbuckle with opposite direction thread portions (not illustrated) configured to mate with opposite thread portions on the separation element, so that rotation of the turnbuckle causes the separation element to lengthen or shorten. Although only one separation element 36 is shown in FIG. 1 associated with one pressure wall, each pressure wall may have its own separation element, and in a preferred embodiment, each pressure wall may have two separation elements, so that the two pressure walls each span between two separation elements. It will be appreciated that this embodiment with a total of four separation elements, one on either side of each pressure wall, provides a more rigid structure, and will provide a more inflexible support for the belts 26, 26′ so that slipping of the belts is better controlled or even eliminated.

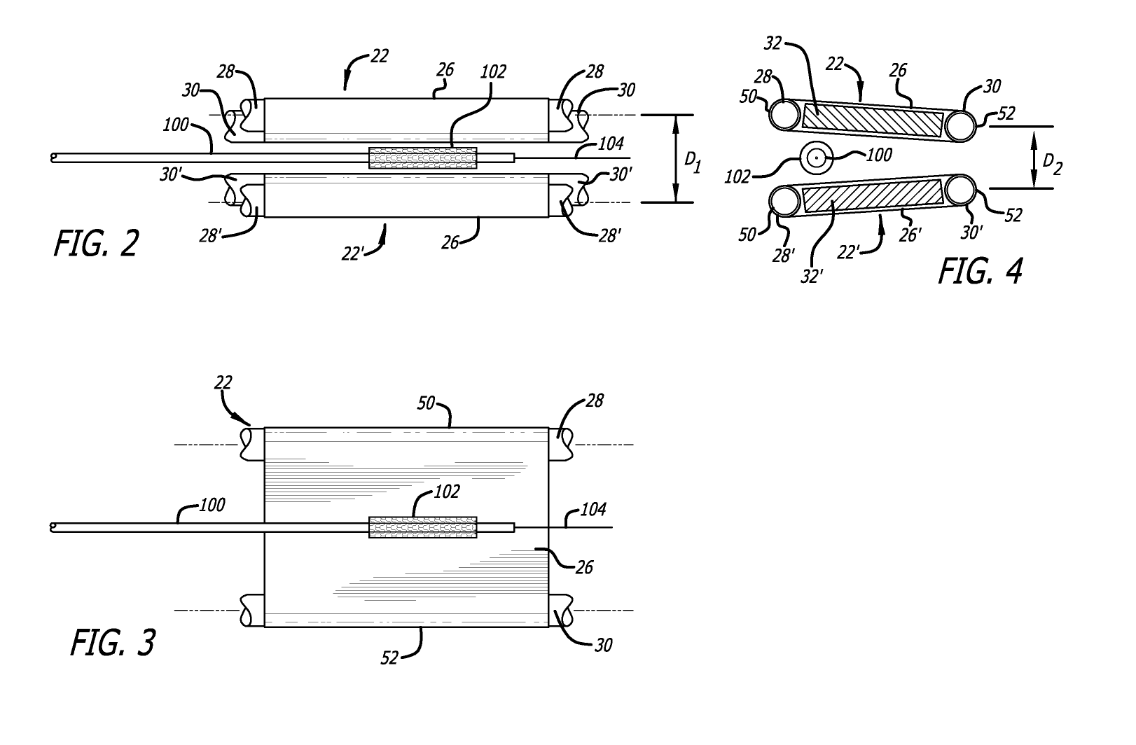

Turning now to use of the device 20 described above, FIGS. 2-4 exemplify how a catheter 100 with a stent 102 positioned over a forward portion of the catheter may be inserted between the two pressure walls. (In FIGS. 2-4 the separation elements of FIG. 1 are not included, for simplicity.) The stent may be positioned over a balloon portion of the catheter so that when the balloon is eventually expanded it will cause the stent to expand. In a preferred embodiment of the invention, a guidewire 104 may be threaded through a central lumen of the catheter 100 when the catheter is placed between the pressure walls. Preferably, the guidewire once threaded through the catheter is placed under tension, so that greater control can be exercised over the stent mounted on the catheter when the guidewire is grasped at either end. The balance of the catheter, not shown in the figures, is preferably laid out to be precisely colinear with the portion of the catheter that is between the pressure walls as shown in FIG2. 2-4, so that the entire catheter may be rotated about a central axis of the catheter. Preferably the catheter is rotated about the guidewire when the guidewire is under tension which provides a suitable support to hold the catheter in a precisely linear configuration and which allows movement of the catheter to be conveniently effected through movement of the tensioned guidewire.

Once the catheter 100 with mounted stent 102 is thus positioned between the pressure walls 22, 22′, rotational power is applied to one or more of the bars to commence rotation of the belts 26, 26′, each belt rotating in the same direction, either clockwise or anticlockwise. It will be appreciated that, when the belts rotate in the same direction, portions of the belt in contact with the stent will move in opposite directions. The guidewire under tension is moved slowly downwardly so that the forward portion of the catheter is moved downwardly between the pressure walls 22, 22′ until the distance between the walls at the level of the stent is equal to the diameter of the stent mounted on the catheter. At this point, the movement of the belts begins to rotate the stent. In order to preserve the structural integrity of the balloon within the stent, the entire catheter is also rotated on its axis about the tensioned guidewire at the same angular velocity as the stent. As the stent rotates, the guidewire is slowly lowered in relation to the crimping device 20, so that the stent is forced into an ever narrower space between the pressure walls. Constant rotation of the stent between the pressure walls with ever decreasing spacing causes the outer diameter of the stent to slowly reduce while the elements making up the stent are plastically deformed to permit the stent to assume a new smaller diameter. Because the catheter is rotated at the same angular velocity as the stent, the balloon rotates with the stent and no frictional shear is applied to the balloon material by the rotating stent.

It will be appreciated that, by empirical study and adjustment on a particular type of stent, the spacing at the top of the pressure walls, D1, and at the bottom of the pressure walls, D2, may be set to optimal dimension to allow the stent mounted on the catheter to be introduced freely between the top edges 50 of the pressure walls, and to allow the fully compressed stent to emerge freely below the bottom edges 52 of the pressure walls once the catheter and stent have been moved to a level below the walls. Thus, once the spacing between the pressure walls is optimized for a particular type of stent and catheter combination, the walls may be fixed in position and need not be adjusted to admit a new stent for compression, nor to release a stent after compression.

It will be further appreciated that, when the belts 26, 26′ are rotated under the action of the bars 28, 28′, the tension in the belts alone may not be sufficient to apply a suitable lateral force that acts as a crimping force on a rotating stent. Therefore, the reinforcing blocks 32, 32′ are provided with the purpose of exerting a radially inward force on the stent, while the belts 26, 26′ mainly provide the function of rotating the stent while the stent is being advanced downwardly between the pressure plates 22, 22′. In this way, a radially inward force may be slowly applied to the entire circumference of the stent as it is moved downward between the pressure plates. At the same time, a force vector in the direction of a tangent to the stent circumference is applied by the rotating belts. This tangential force assists in applying plastic deformation to the stent elements, and assists in producing a desired final crimped diameter without having to initially crimp the stent to a diameter less than the desired final diameter. When the stent reaches the end of its travel between the pressure plates and emerges from the bottom of the plates, the stent has been crimped to substantially exactly the desired degree onto the balloon, and the device 20 is ready to receive the next stent and catheter combination for crimping. In a further embodiment, the direction of rotation of the belts on the pressure walls may be sequentially alternated, so that the stent is rotated first one way then the other. This has the effect of more evenly distributing the rotating tangential crimping force on the stent, and reduces any residual circumferential shear distortion applied to the stent by the action of the belts 26, 26′.

An advantage of the present invention is that it applies a resultant inward and tangential crimping force on the stent that is slowly increased as the stent advances downwardly between the pressure walls. From the perspective of the stent, the loading force rotates around the circumference of the stent, applied on two opposite sides of the stent. In a preferred embodiment, the force is applied under rotation one way, followed by rotation the other way to eliminate any resulting circumferential shear distortion. By this rotational method, plastic deformation of the stent is readily achieved without deforming the stent radially inwardly substantially below its final diameter, and tends to overcome problems in the art in which a radially inward crimping force must be applied to a degree that may damage the balloon before the stent is allowed to make some residual elastic recovery to its final crimped diameter.

It will be realized that the foregoing preferred specific embodiments have been shown and described for the purpose of illustrating the functional and structural principles of this invention and are subject to change without departure from such principles. Therefore, this invention includes all modifications encompassed within the spirit and scope of the following claims.

We claim:

1. A device for crimping a stent mounted on a catheter comprising:
two pressure walls positioned adjacent each other and angled toward each other without contacting each other, each pressure wall including:
two rotatable rods spaced apart and parallel to each other;
a flexible belt looped around the rods, the belt being tensioned to provide a planar surface between the rods and to rotate when at least one of the rods is rotated.

2. The device of claim 1, wherein each pressure wall further includes a reinforcing block having a planar surface, the block being positioned within a perimeter of the belt and the planar surface of the block being positioned adjacent the planar surface of the belt.

3. The device of claim 2, wherein the reinforcing block is attached to a supporting element for holding the reinforcing block stationary relative to the device.

4. The device of claim 3, wherein the supporting element is configured to adjust the position of the reinforcing block relative to the device is adjustable.

5. The device of claim 1, wherein the rods are held apart by at least one separation element.

6. The device of claim 5, wherein the at least one separation element is two separation elements, each separation element positioned on either side of the belt.

7. The device of claim 5, wherein the length of the separation element is adjustable.

8. The device of claim 7, wherein the separation element includes a threaded turnbuckle for adjusting the length of the separation element.

9. The device of claim 1, wherein the rods of the two pressure walls are configured to rotate so that the two belts rotate simultaneously in the same rotational direction.

10. A device for crimping a stent mounted on a catheter comprising:
first and second substantially planar pressure walls positioned adjacent each other, each pressure wall including:
a rigid block having at least one planar surface; and
a sheet of flexible material configured to slide over a surface of the rigid block;
wherein the sheet of flexible material of the first pressure wall is configured to slide in a direction opposite to the direction of the material in the second pressure wall, whereby the sheets are configured to impart a rolling motion to a cylindrical stent placed between the walls.

11. The device of claim 10, wherein each pressure wall further includes means for tensioning the sheet of flexible material.

12. The device of claim 10, wherein each pressure wall has a first edge and an opposite second edge, the first edges being closer to each other than the second edges are to each other.

13. A device for crimping a stent mounted on a catheter comprising:
two substantially planar pressure walls positioned adjacent each other and angled toward each other without contacting each other, each pressure wall including:
a flexible belt;
means for rotating the belt under tension.

14. The device of claim 13, further including means for adjusting tension in the belt.

15. The device of claim 13, further including means for rigidly supporting each flexible belt to maintain a portion of each belt in a planar surface.

16. A method of crimping a stent onto a catheter comprising:
mounting a stent in uncrimped condition onto a catheter;
introducing the stent between two opposing walls, each wall having a planar surface, each surface angled toward the surface on the opposing wall such that the distance between the surfaces at a first edge is greater than the distance between the surfaces at an opposite second edge;
causing the planar surfaces to move in opposite directions to each other;
moving the stent in relation to the walls toward the second edge whereby the stent engages with the surfaces and is rotated under the action of a crimping force;
rotating the catheter so that the stent and the catheter do not rotate in relation to each other while the stent is being moved toward the first edge, whereby the stent becomes radially compressed and crimped onto the catheter.

17. The method of claim 16, wherein causing the planar surfaces to move in opposite directions to each other includes rotating a belt on each of the walls in the same rotational direction.

18. The method of claim 17, wherein rotating a belt on each of the walls in the same rotational direction includes rotating each belt clockwise followed by anticlockwise.

19. The method of claim 16, wherein moving the stent in relation to the walls includes threading a guidewire through a central lumen in the catheter, and then moving the guidewire and the catheter in relation to the walls so that the stent follows the movement of the catheter.

20. The method of claim 19, wherein threading a guidewire through a central lumen of the catheter is followed by placing the guidewire under tension, and holding the guidewire under tension when the guidewire and catheter are moved in relation to the walls.

21. A method of crimping a stent onto a catheter comprising:
mounting a stent in uncrimped condition onto a catheter having a guidewire threaded through a lumen in the catheter;
tensioning the guidewire to provide control over movement of the catheter;
moving the guidewire to introduce the stent between two opposing walls, each wall having a planar surface, each surface being angled toward the surface on the opposing wall such that the distance between the surfaces at a first edge is greater than the distance between the surfaces at an opposite second edge;
causing the planar surfaces to move in opposite directions to each other;
moving the guidewire to move the stent in relation to the walls toward the second edge whereby the stent engages with the surfaces and is rotated under the application of a crimping force.

22. The method of claim 21, wherein causing the planar surfaces to move in opposite directions to each other includes rotating a looped belt on each of the walls in the same rotational direction.

23. The method of claim 22, wherein rotating a looped belt on each of the walls includes supporting each rotating belt with a stationary block having a planar surface.

24. The method of claim 22, wherein rotating a belt on each of the walls in the same rotational direction includes rotating each belt clockwise followed by anticlockwise.

25. The method of claim 22, further including rotating the catheter so that the stent and the catheter do not rotate in relation to each other while the stent is being moved toward the second edge, whereby the stent becomes radially compressed and crimped onto the catheter.
